(19) United States
(12) Patent Application Publication
(10) Pub. No.: US 2006/0200082 A1
(43) Pub. Date: Sep. 7, 2006

(54) SIDE RAIL INTRAVENOUS AND ARTERIAL LINE GUARD

(76) Inventor: Amy Stewart, Orange Park, FL (US)

Correspondence Address: Mark James Young, Unit 202, 12086 Fort Caroline Rd., Jacksonville, FL, 32225, US

(21) Appl. No.: 11/306,333

(22) Filed: Dec. 22, 2005

Related U.S. Application Data
(60) Provisional application No. 60/639,119, filed on Dec. 24, 2004.

(51) Int. Cl. A61M 5/32

(52) U.S. Cl. 604/174

(57) ABSTRACT

A line guard includes a polyhedron object having a top side, a bottom side, and a line side. The line side is configured to prevent prevent abutting lines from sliding up along the line side over the top side of the line guard. In one embodiment, an attachment is provided for releasably securing the line guard to a side rail. The attachment may be comprised of adhesive, hook and loop fasteners, belts with buckles or snap closures, mechanical clips, or a tether. Alternatively, the line guard may be pivotally mounted to a side rail using a pivot pin or a hinge.

### RELATED APPLICATION

This application claims the benefit of priority to U.S. Provisional Application No. 60/639,119 filed Dec. 24, 2004, the entire contents of which are incorporated herein and made a part hereof.

### FIELD OF THE INVENTION

This invention generally relates to intravenous and arterial lines, and, more particularly, to a guide for attachment to a side rail for preventing the lines from slipping off the side rail.

### BACKGROUND

Medical treatments for hospitalized patients frequently entail administering therapeutic agents, drugs, medicines, nutrients, and various other substances into a patient's blood stream via intravenous or arterial lines (i.e., tubing). Typically, such lines are draped over side rails of a patient's bed. As patients often receive several such substances concurrently, several lines may extend from respective supply reservoirs, over side rails to a patient. The length of each line is typically significantly longer than necessary to reach from a reservoir to the patient to provide slack and allow movement of the patient relative to the reservoir.

Serious problems ensue when the lines are snagged, kinked or tugged. Movement of the patient and activity around the patient inevitably causes the lines to move. Often the lines will slide off the bed rail and become tangled or snagged on a portion of the bed, nearby equipment or some other object. Occasionally the object causing the tangle or snag is moved, thereby pulling the line from the patient. This may result in pain, trauma, spilled intravenous fluid and blood loss.

Concomitantly, lines may become kinked or otherwise occluded, thereby restricting fluid flow. Any impediment to the free flow of fluid within the lines will decrease the rate of delivery and possibly effectiveness of a therapy.

Various line organizers are known in the art. Such devices are typically attached to side rails and include a plurality of parallel channels for receiving lines. Organizers may also include identifications for each line. While line organizers are useful for securing lines in place, they severely limit mobility of the lines, are cumbersome to use and hold a limited number of lines.

The invention is directed to overcoming one or more of the problems as set forth above.

### SUMMARY

To overcome problems as set forth above, in one aspect of an exemplary embodiment of the invention a line guard is provided. The line guard includes a polyhedron object having a top side, a bottom side, and a line side. The line side has an overhang. The overhang is configured to prevent abutting lines (i.e., lines adjacent to or abutting the line side) from sliding up along the line side over the top side. In one embodiment, an attachment is provided for releasably securing the line guard to a side rail. The attachment may be comprised of adhesive, hook and loop fasteners, belts with buckles or snap closures, mechanical clips, or a tether. Alternatively, the line guard may be pivotally mounted to a side rail using a pivot pin or a hinge.

Thus, an exemplary line guard according to principles of the invention comprises a polyhedron having a top side, a bottom side, and a line side. In one embodiment, the line side has an overhang configured to prevent abutting lines from sliding up along the line side over the top side. However, the line side may also be a substantially planar surface configured to prevent abutting lines from sliding up along the line side over the top side of the line guard.

The exemplary line guard may also include an attachment adapted for releasably securing the line guard to a side rail of a bed. The attachment may be adhesive, hook and loop fasteners, belts with buckles, belts with snap closures, mechanical clips, or a tether. In an alternative embodiment, the line guard may be pivotally attached to a side rail using a pivot pin or hinge.

In another aspect of an exemplary line guard according to principles of the invention, the line guard may include a patient side and a room side opposite said patient side. A visible indicium (e.g., advertisement) may be displayed on one of the sides.

In another aspect of an exemplary line guard according to principles of the invention, the line guard may include a light source, a switch operably coupled to the light source and a power supply operably coupled to the light source. The switch is configured to activate and deactivate the light source. The power supply is configured to supply power to the light source.

In yet another aspect of an exemplary line guard according to principles of the invention, the line guard is comprised of a plastic material. The material may include a colorant in an amount effective to impart a desired color to the line guard. The material may include a thermochromic additive in an amount effective to cause the line guard to change color at a determined temperature. The material may include a phosphorescent additive in an amount effective to adsorb light energy when a source of light is present and to release the absorbed light energy as visible light after the source of light is removed.

### BRIEF DESCRIPTION OF THE DRAWINGS

The foregoing and other aspects, objects, features and advantages of the invention will become better understood with reference to the following description, appended claims, and accompanying drawing, where:

FIG. 1A is a side view of an exemplary line guard with adhesive attachments according to principles of the invention; and

FIG. 1B is a side view of an exemplary line guard with hook and loop attachments according to principles of the invention; and

FIG. 2 is a side perspective view of an exemplary line guard with adhesive attachments according to principles of the invention; and

FIG. 3 is a side view of an exemplary line guard according to principles of the invention installed on a side rail; and

FIG. 4 is a side view of an exemplary line guard according to principles of the invention pivotally installed on a side rail; and

FIG. 5 is a side view of an exemplary line guard adapted to display an advertisement according to principles of the invention; and

FIG. 6 is a side view of an exemplary line guard equipped with a light according to principles of the invention; and

Those skilled in the art will appreciate that Figures are not intended to be drawn to any particular scale; the invention is not limited to the dimensions or proportions shown in Figures; and the Figures are not intended to illustrate every embodiment of the invention. The invention is not limited to the exemplary embodiments depicted in the Figures or the exact shapes, relative sizes, ornamental aspects, dimensions or proportions shown in the Figures.

### DETAILED DESCRIPTION

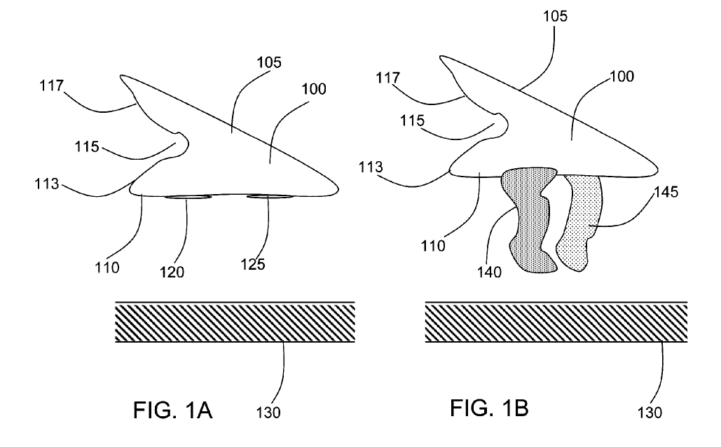

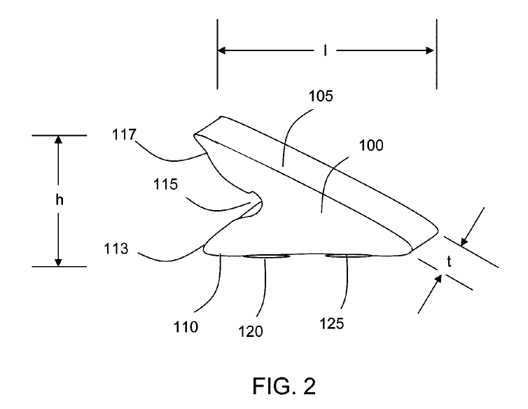

Referring to FIG. 1A, a side view of an exemplary line guard 100 with adhesive attachments 120 and 125 according to principles of the invention is shown. The line guard 100 is comprised of a polyhedron, such as a fin shaped structure, having a top side 105, a bottom side 110, and a line side 113. In the exemplary embodiment shown in FIGS. 1 through 6, the line side 113 (i.e., the side that faces a line) includes a recess 115. Above the recess 115 is an overhang 117 that prevents abutting lines from sliding up along the line side over the top of the guard 100.

The invention is not limited to the exemplary configuration shown in FIGS. 1 through 6. For example, in an alternative embodiment as shown in FIG. 7, the line side 717 may be a planar surface that is either substantially vertical or slanted to prevent abutting lines from sliding up along the line side over the top of the guard 700. Indeed, any shape and configuration effective for preventing abutting lines from sliding up along the line side over the top of the guard 700 comes within the spirit and scope of the invention.

Attachment devices are provided for securing the line guard 100 to an object such as a side-rail of a bed. Preferably the attachment is releasable so that the line guard 100 may be removed for cleaning or disposal after use. Illustratively, the line guard 100 in FIG. 1A includes adhesive 120 and 125 for securing the line guard. The adhesive 120 and 125 may be covered by a removable paper or plastic sheet (not shown). Removal of the cover sheet exposes the adhesive 120 and 125.

FIG. 1B conceptually illustrates a side view of an exemplary line guard with hook and loop fasteners 140 and 145 according to principles of the invention. The hook and loop fasteners 140 and 145 are provided for securing the line guard 100 to an object such as a side-rail of a bed.

Those skilled in the art will appreciate that releasable attachments other than adhesives and hook and loop fasteners may be used without departing from the scope of the invention. By way of example and not limitation the releasable attachments may be comprised of belts with buckles or snap closures, mechanical clips and strings or cords for tying. Additionally, the line guard 100 may be permanently attached to or an integral part of a side rail.

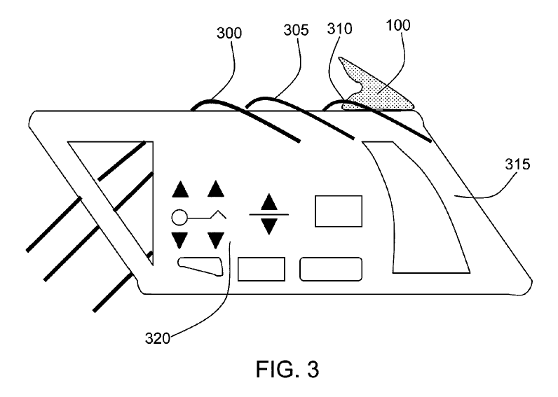

Referring now to FIG. 2, a side perspective view of an exemplary line guard 100 with adhesive attachments 120 and 125 according to principles of the invention is shown. The dimensions and proportions of the line guard 100 are not particularly important, so long as the height (h) of the line side 113 is at least approximately two to three times the outer diameter a line. The thickness (t) of the line guard should be adequate to provide a stable support, at least at the bottom 110. The length (I) of the device should also be adequate to provide a stable support, at least at the bottom 110. Within these parameters, the dimensions of the line guard 100 may vary widely, from fractions of an inch to several inches. Likewise, the proportions of the line guard 100 may also vary.

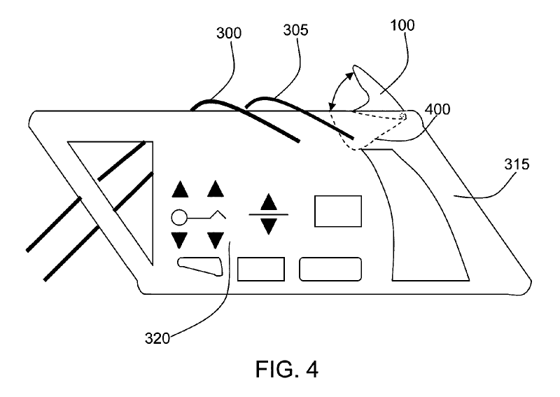

Referring now to FIG. 3, a side view of an exemplary line guard according to principles of the invention is shown installed on a side rail. The line guard may be releasably attached to the side rail 315 using adhesive 120 and 125. The line guard 100 prevents lines 300-310 from sliding off the guarded edge of the side rail 315. Side rail controls 320 are shown for illustrative purposes only, and do not limit the scope of the invention.

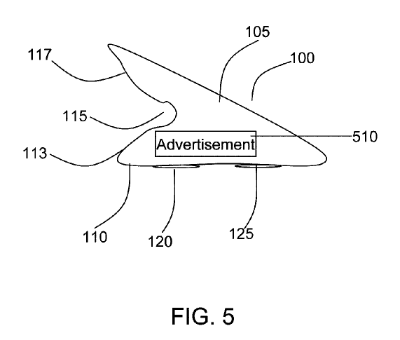

FIG. 4 illustrates an alternative embodiment of an exemplary line guard 100 according to principles of the invention. The line guard 100 is pivotally mounted to the side rail. A cavity 400 is provided, in which the line guard may reside when the line guard is pivoted into a closed position. As shown in FIG. 4, the line guard 100 is in an open position. Other pivot or hinged mountings may be employed without departing from the scope of the invention. [Para 31]While FIGS. 3 and 4 illustrate one line guard 100 installed on a side rail, those skilled in the art will appreciate that additional line guards may be used without departing from the scope of the invention. By way of example, and not limitation, a line guard may be provided at each end of the side rail.

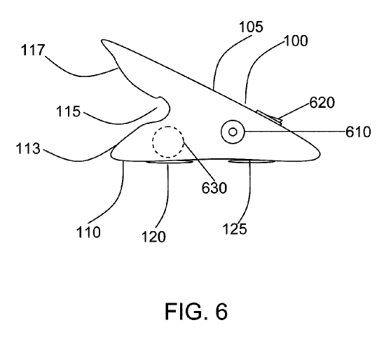

Optionally, indicia may be displayed along one or more surfaces of the line guard 100. By way of example and not limitation, a side (e.g., patient side and/or room side and/or any other visible surface) of a line guard 100 may display information, decorative features or promotional icons, such as an advertisement 510 as conceptually shown in FIG. 5. Thus, the device may provide a unique informational and/or advertising means. For example, in a mode of advertising use, businesses may display their trademarks or service marks along one or more surfaces. During use of the line guard 100, the trademarks or service marks would be conspicuously displayed for all individuals in the vicinity to view. Given the uniqueness of the device and the mode of use, it is believed that the device will draw considerable attention. In heavy traffic settings this could translate into exposure to hundreds or thousands of people resulting in increased name recognition and goodwill.

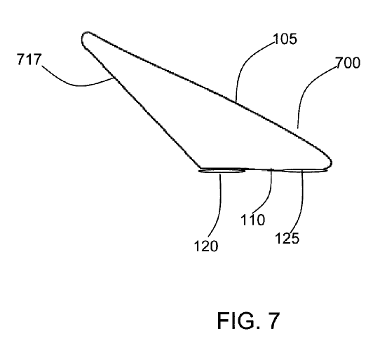

As another option, a line guard according to principles of the invention may be equipped with an electric light source 610, as conceptually illustrated in FIG. 6. The light source may be an incandescent lamp, light emitting diode or any other compatible illumination device. The light may be activated by a switch, such as (for example) a manual switch 620 as shown in FIG. 6. Any circuitry that includes a light source 610, power supply and switch 620 and is configured to enable controllable activation and deactivation of the light source 610 using the switch 620 may be utilized within the spirit and scope of the invention. The power supply may include a battery 630 in a compartment. The battery 630 may be a disposable or rechargeable battery. Alternatively, the line guard may be electrically coupled to a power supply for the bed. Thus, the illuminated line guard may serve as a night light or reading light.

The line guard 100 is preferably comprised of a plastic or polymeric material, such as polyvinyl chloride (PVC), polyethylene, polypropylene, polystyrene, acrylics, cellulosics, acrylonitrile-butadiene-styrene terpolymers, urethanes, thermo-plastic resins, thermo-plastic elastomers (TPE), acetal resins, polyamides, polycarbonates or polyesters. Though many other materials may be used alone or in combination with the aforementioned materials and/or other materials, without departing from the scope of the present invention, preferably the material is relatively inexpensive and durable, easy to use in manufacturing operations and results in an aesthetically acceptable product. The line guard 100 may be produced using any suitable manufacturing techniques known in the art for the chosen material, such as (for example) injection, compression, structural foam, blow, or transfer molding; polyurethane foam processing techniques; vacuum forming; and casting. Preferably the manufacturing technique is suitable for mass production at relatively low cost per unit, and results in an aesthetically acceptable product with a consistent acceptable quality. The material may further include additives to provide desired properties such as desired colors, structural characteristics, glow-in-the dark properties, and/or thermal reactivity (e.g., color changes according to heat).

By way of example and not limitation, the line guard 100 may optionally be formulated to change color when it reaches a predetermined or higher temperature. This can be accomplished by mixing a thermochromic additive to the base material in an amount that is sufficient to achieve a desired color changing range. As an example, a mixture of approximately 5% to 35% (pbw) of Matsui International Co., Inc.'s Chromicolor® concentrate may be introduced to the base material, to provide a plastic structure that visibly changes color at a determined temperature, such as approximately 80 degrees Fahrenheit or higher.

As another alternative, phosphorescent polymer additives, such as aluminate based phosphors, may be added to adsorb light energy and continue to release that energy as visible light after the energy source is removed. Advantageously, such an embodiment provides a line guard 100 that is easy to locate in darkened conditions, making the line guard 100 easy to spot even at nighttime in an unlit room.

While the invention has been described in terms of various embodiments, implementations and examples, those skilled in the art will recognize that the invention can be practiced with modification within the spirit and scope of the appended claims including equivalents thereof. The foregoing is considered as illustrative only of the principles of the invention. Variations and modifications may be affected within the scope and spirit of the invention.

What is claimed is:

1. A line guard comprising a polyhedron having a top side, a bottom side, and a line side, the line side having an overhang, the overhang being configured to prevent abutting lines from sliding up along the line side over the top side.

2. A line guard according to claim 1 further comprising an attachment adapted for releasably securing the line guard to a side rail.

3. A line guard according to claim 2 wherein the attachment is comprised of an attachment means from the group consisting of adhesive, hook and loop fasteners, belts with buckles, belts with snap closures, mechanical clips, and a tether.

4. A line guard according to claim 1 further comprising an attachment adapted for pivotally attaching the line guard to a side rail, said attachment including an attachment means from the group consisting of a pivot pin and hinge.

5. A line guard according to claim 1, said line guard further including a patient side and a room side opposite said patient side, and said line guard further comprising a visible indicium on one of said patient said and said room side, said visible indicium including subject matter from the group consisting of text, a design, a logo, a slogan, a trademark, and a service mark.

6. A line guard according to claim 1 further comprising an light source, a switch operably coupled to said light source and a power supply operably coupled to said light source, said switch being configured to activate and deactivate the light source, and said power supply being configured to supply power to the light source.

7. A line guard according to claim 1, said line guard being comprised of a plastic material.

8. A line guard according to claim 7, said plastic material including a colorant in an amount effective to impart a desired color to the line guard.

9. A line guard according to claim 7, said plastic material including a thermochromic additive in an amount effective to cause the line guard to change color at a determined temperature.

10. A line guard according to claim 7, said plastic material including a phosphorescent additive in an amount effective to adsorb light energy when a source of light is present and to release the absorbed light energy as visible light after the source of light is removed.

11. A line guard comprising a polyhedron having a top side, a bottom side, and a line side, the line side being configured to prevent abutting lines from sliding up along the line side over the top side of the line guard.

12. A line guard according to claim 11 further comprising an attachment adapted for releasably securing the line guard to a side rail.

13. A line guard according to claim 12 wherein the attachment is comprised of an attachment means from the group consisting of adhesive, hook and loop fasteners, belts with buckles, belts with snap closures, mechanical clips, and a tether.

14. A line guard according to claim 11 further comprising an attachment adapted for pivotally attaching the line guard to a side rail, said attachment including an attachment means from the group consisting of a pivot pin and hinge.

15. A line guard according to claim 11, said line guard further including a patient side and a room side opposite said patient side, and said line guard further comprising a visible indicium on one of said patient said and said room side, said visible indicium including subject matter from the group consisting of text, a design, a logo, a slogan, a trademark, and a service mark.

16. A line guard according to claim 11 further comprising an light source, a switch operably coupled to said light source and a power supply operably coupled to said light source, said switch being configured to activate and deactivate the light source, and said power supply being configured to supply power to the light source.

17. A line guard according to claim 11, said line guard being comprised of a plastic material.

18. A line guard according to claim 17, said plastic material including a colorant in an amount effective to impart a desired color to the line guard.

19. A line guard according to claim 17, said plastic material including a thermochromic additive in an amount effective to cause the line guard to change color at a determined temperature.

20. A line guard according to claim 18, said plastic material including a phosphorescent additive in an amount effective to adsorb light energy when a source of light is present and to release the absorbed light energy as visible light after the source of light is removed.
